(19) United States
(12) Patent Application Publication
(10) Pub. No.: US 2012/0046318 A1
(43) Pub. Date: Feb. 23, 2012

(54) METHODS AND COMPOSITIONS FOR TREATING INTERNAL AND EXTERNAL HEMORRHOIDS

(76) Inventor: Moh Samir Amer, Carpinteria, CA (US)

(21) Appl. No.: 12/860,974

(22) Filed: Aug. 23, 2010

(51) Int. Cl. A61K 31/4458; A61P 17/00

(52) U.S. Cl. 514/331

(57) ABSTRACT

The present invention relates to compositions and methods for treatment of internal and/or external hemorrhoids, wherein the treatment includes topically administering to a subject a composition comprising from 0.3% to 0.7% of S-2′-[2-(1-methyl-2-piperidyl)ethyl]cinnamanilide, wherein the S-2′-[2-(1-methyl-2-piperidyl)ethyl]cinnamanilide not only treats the hemorrhoidal symptoms but delays or inhibits the recurrence of hemorrhoids and/or symptoms thereof.

### BACKGROUND OF THE INVENTION

1. Technology Field

The present invention relates generally to the treatment of hemorrhoids, and more particularly, to compositions and methods for treatment of internal and/or external hemorrhoids, wherein the treatment includes a reduced, yet surprisingly effective, amount of iferanserin that not only treats the condition but delays or inhibits the recurrence of hemorrhoids and/or symptoms thereof.

2. Related Art

About 9% of the people in Western countries suffer from some hemorrhoidal symptoms. The percentage increases with age and reaches 50% after age 50. The disease is more prevalent in men than in women and is generally associated with bleeding, hence the term “hemorrhoid.” The disease is aggravated by straining during defecation, heavy lifting and during pregnancy. Such conditions exert physical pressure on the exit veins interfering with the escape of the blood from the hemorrhoid plexus. This is particularly true during defecation of hardened and compact stools. As hemorrhoids progress the trapped blood forms piles (protruding skin folds filled with static and thrombosed blood), first above the pectinate (dentate) line (grade I), then extending below it but return spontaneously (grade II). The piles next become large enough to protrude outside the anus on straining and have to be manually replaced (grade III). Finally, the protruding piles become irreversible and prolapse (protrude) permanently and cannot be repositioned (grade IV). Discomfort usually accompanies manipulation of the area, particularly during defecation.

There are two main types of hemorrhoids, that being, internal and external. Internal hemorrhoids originate above the dentate line and are characterized by bleeding and irritation but usually do not cause pain. Since the area above the dentate line does not contain sensory nerves, no pain generally accompanies internal hemorrhoids. By contrast, external hemorrhoids originate below the dentate line (usually at the anal verge) and are very painful. External hemorrhoids do not generally bleed since the dermis at the verge is thicker.

Hemorrhoid disease is a varicose dilatation of the blood vessels in the superior or inferior hemorrhoidal plexus, resulting from persistent increase in pressure brought about by the constriction of downstream colonic veins. Occlusion brought about by platelet aggregation and thrombus formation also contributes to the symptoms of hemorrhoids by increasing blood stasis and tissue congestion. This results in masses of dilated vessels situated near the anal sphincter, usually above the dentate line. Such masses underlie the uncomfortable feeling of fullness and throbbing and result in difficulty and pain while defecating.

In slow-moving blood, blood platelets tend to clump and aggregate and release their contents of vaso-constrictive and platelet aggregating agents (the platelet release reaction) of which the most prominent is serotonin (5-hydroxytryptamine, 5-HT) which mediates edema and inflammation and leads to local irritation and pain. The resultant increased platelet aggregation and vaso-constriction cause greater elevation of venous pressure that further decreases blood flow. This establishes a vaso-constrictive cycle resulting in diminished blood perfusion in the affected tissues associated with swelling, irritation and pain. These symptoms are the hallmarks of hemorrhoids. The different 5-HT receptors mediate different functions of 5-hydroxytryptamine. The different 5-HT receptors also appear to be structurally different when cloned and present widely different binding characteristics. 5-HT_{2A} receptors are located in the vasculature and mediate the vasoconstrictive and platelet aggregatory effects of 5-HT.

It was previously discovered in U.S. Pat. No. 5,780,487, the contents of which are incorporated by reference herein, that treatment of hemorrhoids in animals could be accomplished by the administration of a 5-HT₂ receptor antagonist, that being iferanserin, and more specifically, the S enantiomer of 2′-[2-(1-methyl-2-piperidyl)ethyl]cinnamanilide hydrochloride (MPEC). S-MPEC is a selective antagonist at peripheral serotonin 2A (5-HT_{2A}) receptors. As such, it is capable of antagonizing 5-HT_{2A}-mediated smooth muscle vasoconstriction and platelet aggregation, both of which are involved in the formation and maintenance of hemorrhoids.

Although previous use of S-MPEC has been found effective, it would be advantageous to determine a reduced effective dose of the S-MPEC compound while still providing the same therapeutic efficacy.

### SUMMARY OF THE INVENTION

The present invention provides for a surprisingly effective composition for the treatment of both internal and external hemorrhoids, wherein the compositions have a reduced amount of S-2′-[2-(1-methyl-2-piperidyl)ethyl]cinnamanilide hydrochloride having the following structural formula:

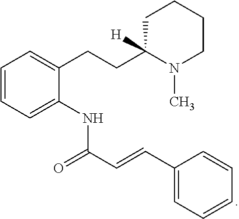

In one aspect, the present invention provides for a composition comprising from about 0.3% to about 0.7% concentration of S-2′-[2-(1-methyl-2-piperidyl)ethyl]cinnamanilide hydrochloride, and preferably a concentration of about 0.5%, wherein the S-2′-[2-(1-methyl-2-piperidyl)ethyl]cinnamanilide hydrochloride is substantially free or devoid of the (R) enantiomer. The S-2′-[2-(1-methyl-2-piperidyl)ethyl]cinnamanilide hydrochloride may be mixed with a pharmaceutically acceptable cream, gel, paste, lotion, ointment, emulsion, foam, liquid or a combination thereof.

In another aspect, the present invention provides for a homogenous composition for treating internal and/or external hemorrhoids, the composition comprising:
(a) S-2′-[2-(1-methyl-2-piperidyl)ethyl]cinnamanilide hydrochloride in an amount from 0.3% to 0.7%;
(b) liquid paraffin in an amount form about 5% to 20%;
(c) cetanol in an amount from about 2% to about 10%; and
(d) white vaseline to provide for 100%.

In yet another aspect, the present invention provides for a method of treating internal and/or external hemorrhoids, the method comprising:
(a) administering a topical composition to the anorectal region of a subject in need of such treatment, wherein the composition comprises from about 0.3% to 0.7% concentration of S-2′-[2-(1-methyl-2-piperidyl)ethyl]cinnamanilide hydrochloride, wherein the S-2′-[2-(1-methyl-2-piperidyl)ethyl]cinnamanilide hydrochloride is substantially free or devoid of the (R) enantiomer; and
(b) repeating step (a) for a period ranging from one (1) day to thirty (30) days, more preferably from three (3) days to fourteen (14) days, and most preferably, from seven (7) days to fourteen (14) days.

In still a further aspect, the present invention relates to a method of delaying or inhibiting recurrence of internal and/or external hemorrhoids, the method comprising:
administering a topical composition to the anorectal region of a subject in need of such treatment, wherein the composition comprises from about 0.3% to about 0.7% concentration of S-2′-[2-(1-methyl-2-piperidyl)ethyl]cinnamanilide hydrochloride for a sufficient period of time to reduce or inhibit recurrence of symptoms due to hemorrhoids.

Preferably, the recurrence of symptoms is delayed at least three (3) days to about two (2) months. More preferably, there is total inhibition of any recurrence of hemorrhoidal symptoms.

In another aspect, the present invention provides for a single dose applicator device comprising an ointment, wherein the ointment comprises from about 0.3% to 0.7% concentration of S-2′-[2-(1-methyl-2-piperidyl)ethyl]cinnamanilide. Preferably, the amount is 0.5% and effective to delay or inhibit the recurrence of symptoms due to hemorrhoids.

In yet another aspect, the present invention provides for molecules having therapeutic effects on hemorrhoidal symptoms, wherein the molecules have the following structural formulas:

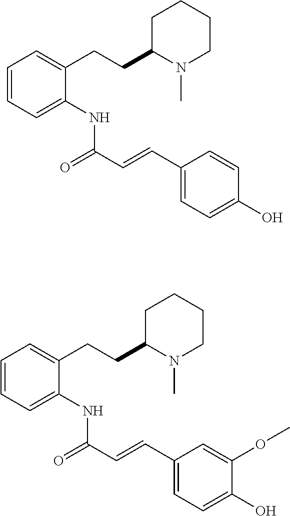

and wherein the compounds are formed in vivo due to the metabolism of iferanserin by a treated subject or in the alternative the compounds are synthesized ex vivo or in a laboratory and formulated for administering to a subject for the treatment of hemorrhoidal symptoms.

In a still further aspect, the present invention provides for a composition comprising S-2′-[2-(1-methyl-2-piperidyl)ethyl]cinnamanilide hydrochloride and a synthesized metabolite as described in FIG. 1.

In a final aspect, the present invention provides for a kit comprising a sufficient amount of single use applicators to treat hemorrhoidal symptoms, wherein each applicator comprises a composition comprising from about 0.3% to about 0.7% concentration of S-2′-[2-(1-methyl-2-piperidyl)ethyl]cinnamanilide hydrochloride, wherein the S-2′-[2-(1-methyl-2-piperidyl)ethyl]cinnamanilide hydrochloride is substantially free or devoid of the (R) enantiomer. Preferably, the kit includes a sufficient number of the single use applicators to treat the hemorrhoidal symptoms over a period from 1 to 14 days.

Other aspects and advantages of the invention will be more fully apparent from the ensuing disclosure and appended claims.

### BRIEF DESCRIPTION OF THE FIGURES

FIG. 1 shows a metabolic scheme for the metabolism of the (S) enantiomer of 2′-[2-(1-methyl-2-piperidyl)ethyl]cinnamanilide hydrochloride.

FIG. 2 is a graph showing that the 0.5% dose group provided the most consistent improvement in hemorrhoidal symptoms including: bleeding, pain, ease of defecation, swelling and size.

FIG. 3 is a bar graph shows the percentage of change in specific symptoms relative to the % percent concentration, wherein the 0.5% concentration showed the greatest reduction in all the symptoms.

FIG. 4 is a graph showing that symptoms in the group using the 0.5% concentration started improving on day one (1), peaked on day seven (7) and were maintained until day fourteen (14).

FIG. 5 is a bar graph showing the reduction in hemorrhoid size at day 28 relative to the different concentrations. Again, the 0.5% dose level was the most effective on the primary efficacy parameters.

FIG. 6 is a bar graph showing the number of patients experiencing return of hemorrhoidal symptoms within forty-five (45) days after completion of a fourteen (14) day study.

FIG. 7 is a bar graph showing the number of patients ceasing bleeding for a minimum of three (3) consecutive days on the specific days of the study.

FIG. 8 is a bar graph showing the number of patients ceasing itching for a minimum of three (3) consecutive days on the specific days of the study.

FIG. 9 is a bar graph showing the number of patients ceasing pain for a minimum of four (4) consecutive days on the specific days of the study.

### DETAILED DESCRIPTION OF THE INVENTION

Hemorrhoids, which are characterized by the inflammation and swelling of veins around the anus or lower rectum, can cause bleeding, itching, pain and difficulty defecating.

Iferanserin is a powerful and selective 5-HT_{2A} receptor antagonist. Since it does not cross the blood/brain barrier (except at extremely high doses in animal studies), iferanserin represents the first of a class of 5-HT_{2A} receptor antagonists that acts primarily in the periphery. Iferanserin is particularly selective to human colonic venous 5-HT_{2A} receptors, although there is data supporting activity at both 5-HT_{2B} and 5-HT_{2C}. In all systems studied, iferanserin selectively blocked 5-HT_{2A} receptors with little effect on 5-HT-1, dopamine or adrenergic receptors. More importantly, iferanserin effectively antagonized the constrictive effects of 5-HT on human colon veins. It also blocked the platelet aggregation effects of 5-HT on human platelets. Since 5-HT is released by the blood platelets in static and pooled blood, selective peripheral 5-HT_{2A} receptor antagonists would be expected to have built-in tissue selectivity, i.e., will act only where there is vaso-constriction and platelet aggregation mediated by 5-HT. Therefore, the effects of iferanserin should be limited only to those areas where there is stasis and pooled blood caused by serotonin.

The present invention comprising iferanserin, and preferably the (S) enantiomer, as the active agent, is believed to be more efficacious and/or less invasive than conventional hemorrhoid therapies. Iferanserin has the ability to significantly reduce bleeding, pain and itchiness with minimal adverse effects. Further, other effects of iferanserin include a local anesthetic effect that is equivalent to that of lidocaine and also exhibits an anti-inflammatory effect, thereby reducing edema is areas experiencing the effects of hemorrhoids.

This invention comprises therapeutic compositions comprising S-2′-[2-(1-methyl-2-piperidyl)ethyl]cinnamanilide (S-MPEC) or a pharmaceutically acceptable acid salt thereof, wherein the S-MPEC is substantially free of the R isomer. The term “substantially free” is intended to cover mixtures containing from about 0.0001% to about 10% of the R isomer (stereoisomer). Preferably, the substantially free molecule contains no more than about 4%, and more preferably, no more than about 1% of the R-MPEC impurity. Most preferably, the S-MPEC is 100% pure and devoid of the (R) enantiomer.

The present invention also provides other active ingredients that may be combined with S-2′-[2-(1-methyl-2-piperidyl)ethyl]cinnamanilide for treatment of internal and/or external hemorrhoids. For example, the S-2′-[2-(1-methyl-2-piperidyl)ethyl]cinnamanilide compound can be combined with antibiotics, antifungals, antivirals, corticosteroids (e.g., hydrocortisone or triamcinolone), non-steroidal anti-inflammatory drugs (including specifically diclofenac or COX-2 inhibitors such as nimesulide or piroxicam), or salicylates (e.g., salsalate or sulfasalazine). Such compositions may be applied to the anal region at effective and non-toxic dosages for treatment of the symptoms of hemorrhoids.

It is preferable that any composition described herein is administered at effective and non-toxic dosages, such that the subject experiences relief from symptoms in the absence of any undesirable side effects. Dosage in individual patients—regarding the amount to be applied with each application, and the frequency of application, should take into account the physical dimensions of the area to be treated, and rate of absorption and metabolism of all ingredients that are absorbed into the systemic circulations, the stage of the disorder to be treated, and what other pharmacological agents are administered concurrently.

Compositions in the form of ointments, creams, gels, pastes, suppositories, liquids, emulsions, foams, aerosols, semisolid powders, or any other form suitable for topical administration are acceptable compositions for the topical treatment of the anorectal pain.

In the preparation of the present invention, suitable carriers can be chosen depending on the dosage forms and include, but are not limited to, hydrocarbons such as vaseline, liquid paraffin, and plasticized hydrocarbon gel (plastibase); animal and vegetable oils such as medium-chain fatty acid triglyceride, lard, hard fat, and cacao oil; higher fatty acid and alcohols and esters thereof such as stearic acid, cetanol, stearyl alcohol, and palmitic acid isopropyl; water-soluble bases such as Macrogol (polyethylene glycol), 1,3-butylene glycol, glycerol, gelatine, white sugar, and sugar alcohol; emulsifiers such as glycerine fatty acid ester, stearic acid polyoxyl, and polyoxyethylene/or curing castor oils; thickeners such as water-dispersible gums, carboxyvinyl polymers, methyl cellulose, sodium carboxymethyl cellulose, and alginates; and preservatives such as paraoxybenzoic acid esters. The preparation of the present invention can be prepared with the aforementioned carriers by methods well-known to those skilled in the art. In addition to said carriers, additives such as stabilizers, pH adjusting agents, diluents, surfactants, neutralizers, antiseptics, germicides, and antioxidants are, if necessary, used. The external preparation of the present invention can be applied to the tropical wound site by conventional methods.

Some of the compositions listed above (e.g. creams, lotions, ointments and gels) may be used in the inventive compositions as thickening agents to create highly convenient dosage forms. Thickened solutions permit release of the active compound to the skin or tissue upon or following application. These forms are advantageously employed to lessen the runoff from the skin or tissue that can occur with more fluid (less viscous) formulations. Importantly, they also permit more sustained contact of the active compound(s) and any penetration enhancer with the treated surfaces, thus permitting an enhancement of the speed of delivery of the active compound(s) to the inflamed tissues and providing more accurate and controllable dosing.

The base cream for use as an ointment may include components such as petrolatum album, liquid petrolatum, beeswax, liquid paraffin, Cetanol, and/or water, wherein the components are mixed until homogenous. More preferably, the base cream of the present invention includes the active agent S-MEPC, liquid paraffin, cetanol and white vaseline, wherein the active agent is in an amount from about 0.3% to 0.7%, the liquid paraffin is in an amount form about 5% to 20%, the cetanol is in an amount from about 2% to about 10% and the white vaseline makes up the remainder to 100%. Most preferably, a base cream or ointment comprises or consists of 0.5% of S-MEPC, 10% of liquid paraffin, 6% of cetanol and the remaining amount of 83.5% of white vaseline.

Dosage may include a single dose applicator wherein approximately 6 to 14 mg of iferanserin (S-MEPC) in the form of two (2) grams of 0.3% to about 0.7% ointment is placed within the applicator. Preferably, the amount of iferanserin is 10 mg thereby providing a 0.5% concentration.

Applicators, such as dispensing tubes, syringes, etc., containing a single dose can provide convenient dosage forms. Squeeze tubes for lotions and ointments may be employed for topical application of the composition for liquids ranging from those of water-like viscosity or the more viscous formulations of thickened compositions and the like.

In treatments according to the invention, an amount of the composition of the invention is contacted with or applied to the affected anal area or proximate thereto such that an effective amount of the antagonist is administered. For example, an ointment composition of the invention can be applied topically at each application to the external anus and to the distal anal canal with the finger or an applicator. As an illustrative alternative, the medication can be delivered rectally as a suppository. The medication can be applied in this fashion, for example, twice daily in the form of an ointment or one or more times daily in the case of the suppository.

The present invention will now be illustrated by the following non-limiting examples.

### Example 1

Preparation and Confirmation of S-MPEC having the following structural formula:

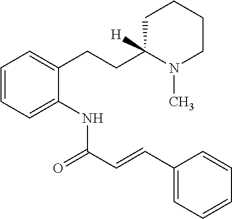

S-MPEC Chemical Process

(A) 2-(o-Nitrostyryl)-1-Methylpyridinium Iodide (NSMP-I)

To a 50 L round bottomed flask was added 2-nitrobenzaldehyde (3,500 g. 23.2 moles), 2-picoline (3.2 L., 32.8 moles) and acetic anhydride. The mixture was stirred efficiently under an inert atmosphere (nitrogen or another inert gas) and heated to reflux for 27 hrs. The mixture was cooled to less than 100° C., for safe handling, and quenched in a suitable vessel equipped with external cooling and efficient stirring on 10.5 Kg. of ice. The pH was adjusted to 11 with 45% aqueous sodium hydroxide at a rate to keep the temperature below 50° C. After cooling to 20° to 30° C., the granular solid was collected by filtration, washed well with water. Yield 6572 g. of crude 2-(o-nitrostyryl)pyridine (NSP).

This solid was transferred to a 50 L, round bottomed flask, dissolved in acetone (14 L) and iodomethane (2.94 L., 47.7 moles) (quaternizing methylating agent) was added. (Other such (alkylating) agents may be used, generally having the formula CH₃X, wherein X is an anion such as sulfate, methyl sulfate, halide (Cl, Br, I), etc.). The mixture was heated to reflux under an inert atmosphere (nitrogen or another inert gas) for 18 hrs. After cooling to 20° C. the precipitate was collected by filtration and washed with acetone or a 1:1 mixture of acetone: ethyl acetate (3×3.5 L). Drying to constant weight at 50° C. to 60° C. yielded 6,839 g. (80%) of NSMP.I.

(B) RS-2-(o-Aminophenethyl)-1-Methylpiperidine, Hydroiodide (RS-APEMP.HI)

In a 5 gallon reactor, a solution of NSNP.I (935 g., 2.5 moles) in methanol (14 L.) was reduced in a hydrogen atmosphere (Psi. 55) in the presence of Pt/C (5 or 10%, 98 g.). After removal of the catalyst and evaporation of the filtrate in the usual manner, the residue was dissolved in hot methanol (2.8 L.). Ethyl acetate (2.8 L) was added to the hot mixture to induce crystallization, yield 516.3 g. (59%) of RS-APEMP.HI.

(C) 5-[2-(o-Aminophenethyl)-1-Methylpiperidine Dibenzoyl-L-Tartrate] (S-APEMP.DBLT)

A solution of RS-APEMP.HI (516 g., 1.5 mole) ethyl acetate (5.5 g.) (or other low boiling water immiscible solvent such as benzene, toluene etc.) was extracted with 5% aqueous sodium hydroxide to liberate the free base (organic phase), washing the organic phase with water, drying over a suitable drying agent (such as anhydr. sodium sulfate, magnesium sulfate, potassium carbonate etc.). After separating the solvent from the drying agent the solution was evaporated in vacuo and the residual RS-APEMP free base was dissolved in methanol and a solution of dibenzoyl-L-tartaric acid (540 g., 1.5 moles) in methanol (2.3 L.) was added. The mixture was held overnight at room temperature. The crystalline precipitate was collected and recrystallized from methanol (3.4 L.); yield 246 g. of S-APEMP.DBNLT. (28.6%, wt; 57.2% of the S-APEMP).

(D) S-2′-[2-(1-Methyl-2-Piperidyl)ethyl]Cinnamanilide (S-MPEC)

A solution of S-APEMP.DBLT (287 g, 0.5 mole) in ethyl acetate (3.2 L.) (or other low boiling water immiscible solvent) was extracted with 7.5% aqueous sodium bicarbonate (3.2 L.) to liberate the S-APEMP. After a water wash and drying over a suitable drying agent the solvent was removed in vacuo. The oily residue, S-APEMP, was dissolved in ethyl acetate (1.0 L.) and anhydrous potassium carbonate (412 g, 3.0 moles) (or other suitable acid acceptor such as triethyl amine, pyridine etc.) was added. Cinnamoyl chloride (143 g., 0.7 mole) in 700 ml. of ethyl acetate was added slowly. After the initial reaction, the mixture was refluxed for 14 hrs. After cooling to room temperature the mixture was extracted with water (1.7 L.) and dried over a suitable drying agent. After removing the drying agent the solvent was removed in vacuo and the residue was dissolved in hot ethyl acetate (280 ml.) and allowed to slowly cool to room temperature; filtration yielded S-MPEC, (136 g., 79% yield). Analysis: Calcd. For C, H, N: C, 79.27; H, 8.10; N, 8.04. Found: C, 79.27; H, 8.06; N, 8.07. HPLC (chiral) purity: 99.5%, [∞]_{D25°}=−46° (c=0.01,EtOH); Melting point: 128° C.

### Example 2

Iferanserin Metabolism

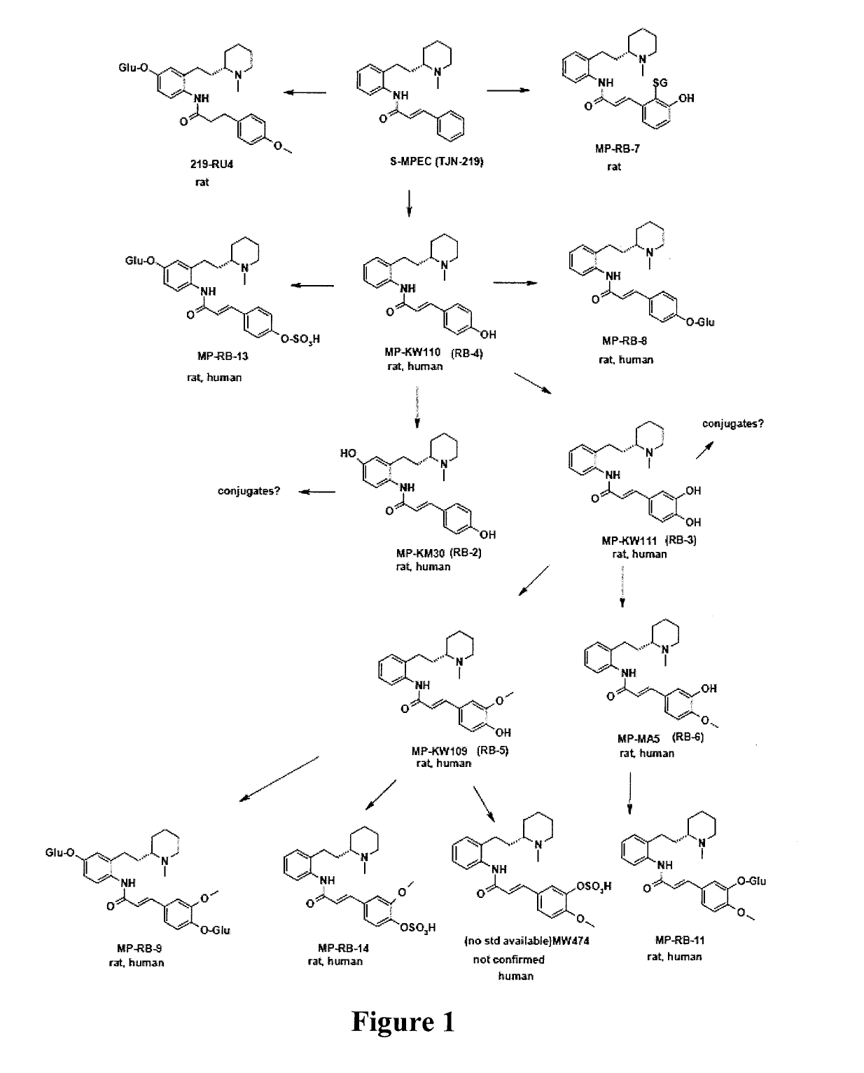

Metabolism of iferanserin was investigated in the rat bile, urine and plasma following administration of [¹⁴C] iferanserin using spectroscopic means. Based on the profile in the rat urine and bile, it is evident that iferanserin is extensively metabolized prior to excretion. An overall metabolic scheme is shown in FIG. 1, based on what has been characterized in the urine from rat and human, and in the bile from rat. It appears that the primary reaction is oxidation on the aromatic rings. This may be followed by O-methylation and/or conjugation with glucuronic acid, sulfate or glutathione. The main metabolites are MP-KW109, MP-KW 110 and MP-MA5. Importantly, both MP-KW 109, MP-KW110 were found to have 5-HT_{2A} antagonistic activity in vitro.

Three groups of 6 healthy male volunteers (a total of 18) were administered, via anal administration, a single dose of 10, 20 or 40 mg iferanserin in the form of two (2) grams of 0.5%, 1.0%, or 2.0% ointment. Venous blood at 0.5, 1, 1.5, 2, 3, 5, 8, 12, and 24 h was sampled, and plasma was prepared (heparin sodium as the anticoagulant). Plasma concentrations of iferanserin and metabolites (KW109, KW110 and MA5) were quantitated using a validated LC/MS/MS method. The pharmacokinetics parameters of iferanserin and its three metabolites in human plasma are listed below in Table 1.

Summary of plasma pharmacokinetics in healthy volunteers following
a single intra-rectal dose of 10, 20, 40 mg iferanserin

| | 10 mg | 10 mg (S-03) | | |
|---|---|---|---|---|
| | (except | (CYP2D6 | | |
| | S-03) | PM*) | 20 mg | 40 mg |
| iferanserin | | | | |
| AUC_{0-inf} | 57.4. ± 26.6 | 1000 | 76.4 ± 47.5 | 188 ± 127 |
| (ng · h/mL) | (26.6-86.1) | | (18.3-126) | (62.5-412) |
| Mean ± SD | | | | |
| (range) | | | | |
| Cₘₐₓ (ng/mL) | 10.8 | 35.1 | 22.1 | 40.9 |
| tₘₐₓ (h) | 1.8 | 5.0 | 1.5 | 1.8 |
| t_{1/2} (h) | 2.0 | 16.7 | 1.9 | 3.0 |
| KW109 | | | | |
| AUC_{0-inf} | 132 | BLQ** | 316 | 549 |
| (ng · h/mL) | | | | |
| Cₘₐₓ (ng/mL) | 11.3 | | 39.2 | 49.7 |
| tₘₐₓ (h) | 4.2 | | 3.2 | 4.8 |
| t_{1/2} (h) | 8.4 | | 6.0 | 7.3 |
| KW110 | | | | |
| AUC_{0-inf} | 135 | BLQ** | 254 | 715 |
| (ng · h/mL) | | | | |
| Cₘₐₓ (ng/mL) | 11.7 | | 38.7 | 70 |
| tₘₐₓ (h) | 4.0 | | 2.8 | 3.3 |
| t_{1/2} (h) | 6.5 | | 5.8 | 5.4 |
| MA5 | BLQ** | BLQ** | BLQ** | BLQ** |

*Poor Metabolizer
**Below Level of Detection

AUC and Cₘₐₓ of the parent compound showed extensive fluctuation, thus a clear dose-proportionality could not be established. But the mean values were approximately linearly related to the dose, except for subject S-03, who was identified as a CYP2D6 poor metabolizer (type D, CYP2D6*5). Apparent terminal half-life of iferanserin was 1.9-3.0 h, and that of metabolites MP-KW 109 and MP-1 10 was 5.4-8.4 h. The values for MA5 were below the limit of quantitation. Interestingly, exposure to metabolites KW 109 and KW 1 10, at 0.5% concentration of Iferanserin, showed a half-life that is 3-4 fold that of iferanserin, except in subject S-03. It is possible that the metabolites provide extended activity providing for extended timing between dosages.

Subject S-03 showed a higher Cₘₐₓ (3-fold) and AUC (17-fold), a delayed tₘₐₓ (5.0 h), and a prolonged half-life (16.7 h). Simulation for a twice-a-day (b.i.d.) multiple dose regime (10 mg) showed that steady state is likely reached in 4-5 days with a projected C_{max,ss} of 90 ng/m. Plasma levels of the three metabolites analyzed for the 5-03 subject were below the limit of quantitation in this particular subject.

### Example 3

Improvement of Hemorrhoidal Symptoms

It is speculated that internal hemorrhoids become symptomatic when the supporting structures become disrupted and the vascular anal cushions prolapse. Hemorrhoids occur more frequently in people with constipation who have hard, infrequent stools. Symptoms attributed to hemorrhoids include bleeding, protrusion, itching and pain. Most hemorrhoidal symptoms arise from enlarged internal hemorrhoids. Bleeding is the most common presenting symptom. Abnormal swelling of the anal cushions, stretching of the suspensory muscles, and dilation of the submucosal arteriovenous plexus result in the prolapse of upper anal and lower rectal tissue through the anal canal. This tissue is easily traumatized, leading to bleeding. The blood is typically bright red due to the arterial oxygen tension caused by arteriovenous communications within the anal cushions. Painless bleeding is usually seen on the toilet tissue or dripping into the toilet at the end of defecation. Sometimes the bleeding can be more substantial, and the blood can accumulate in the rectum with the passage of dark blood or clots. When hemorrhoids prolapse, blood or mucus may stain a patient's underwear, and the mucus against the anal skin may lead to itching. The aforementioned mass of dilated vessels near the anal sphincter underlie the uncomfortable feeling of fullness and throbbing, may bleed, and result in difficulty and pain while defecating. These symptoms were evaluated in this testing regime.

A randomized, double-blind dose study of iferanserin (S-MPEC) was conducted using 0.25%, 0.5%, 1.0% concentrations, wherein the respective dose was applied intra-rectally twice a day (b.i.d) for 2 weeks in patients with hemorrhoids. The primary endpoint of this trial was the improvement in hemorrhoidal symptoms and objective findings including swelling and hemorrhoidal size. Secondary endpoints were subjective symptoms of bleeding, pain, anal discomfort, difficulty in defecation and prolapse rated on a visual analog scale.

Efficacy Results:

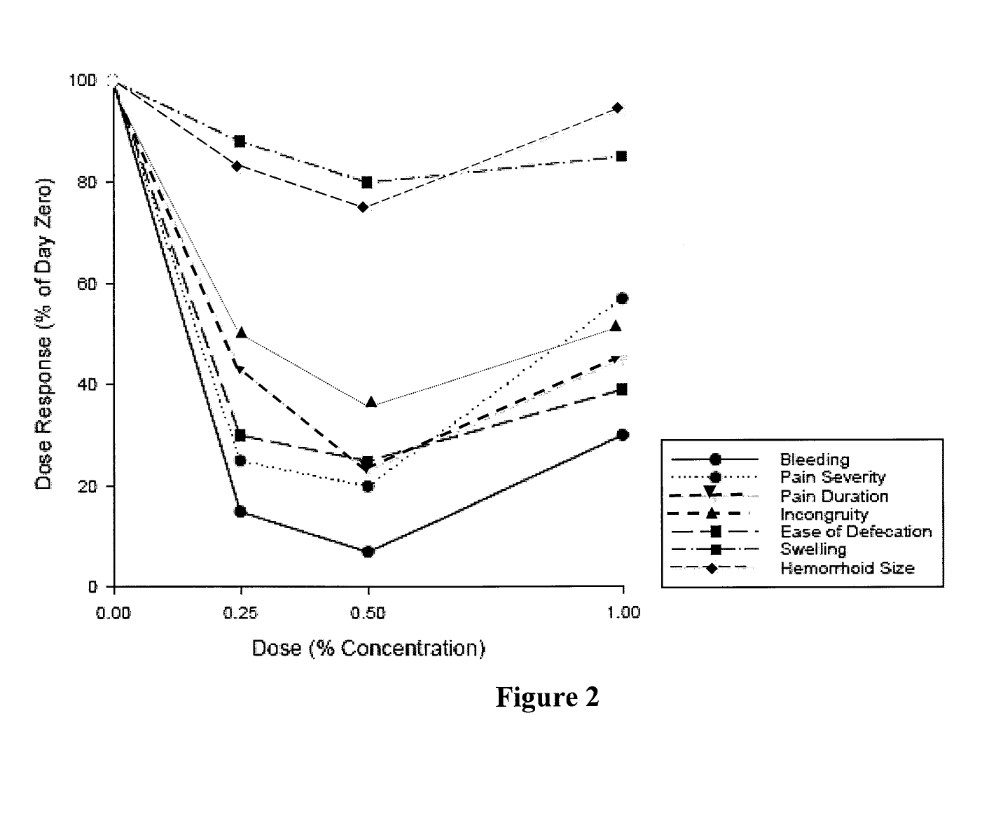

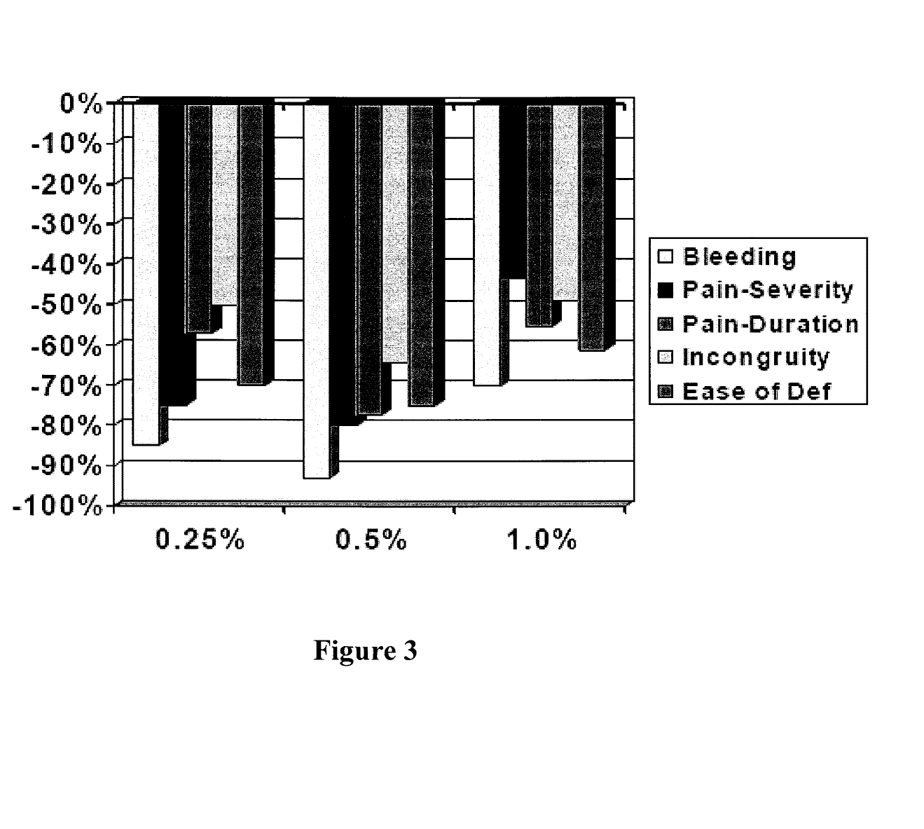

Significant reductions in bleeding, pain severity and duration and ease of defecation were seen after only one day of application. Following two weeks of drug application, virtually all the symptoms of hemorrhoids disappeared and the size of the hemorrhoids themselves were significantly (statistically) reduced. It should be noted that the 0.5% dose group (versus 0.25 and 1% dose groups) provided the most consistent improvement in bleeding, pain severity and duration, ease of defecation, swelling and size as shown in FIGS. 2 and 3. According to the data obtained from the completed studies, efficacy should be established for most patients within 1-2 days of therapy. However, using a 7-day therapy gives the patients more time to achieve the desired response. Additionally, the completed study demonstrates that most of the drug effects observed were complete, or essentially complete, within seven days of drug application, hence a seven day endpoint appears optimal. The adverse events reported were gastro-intestinal system disorders such as “abdominal discomfort” and “diarrhea,” all of them were mild and the patients recovered without any treatment.

### Example 4

The S isomer of MPEC in doses of 0.25%, 0.5% and 1.0% cream was used for symptomatic internal and mixed internal/external hemorrhoids and administered twice a day for 14 days. Seventy two patients were enrolled; 68 evaluable for analysis: 0.25% (23 pts), 0.5% (24 pts), 1.0% (21 pts). A graded assessment method used the following methods: if a value after treatment was smaller (−) it meant “improved,” if a value after treatment was equal (=), it meant “no change” and a value that was greater (+) after treatment it meant “deteriorated.” There was significant change in ease of defecation (week one) between dose groups; but no other symptom dose difference was detected.

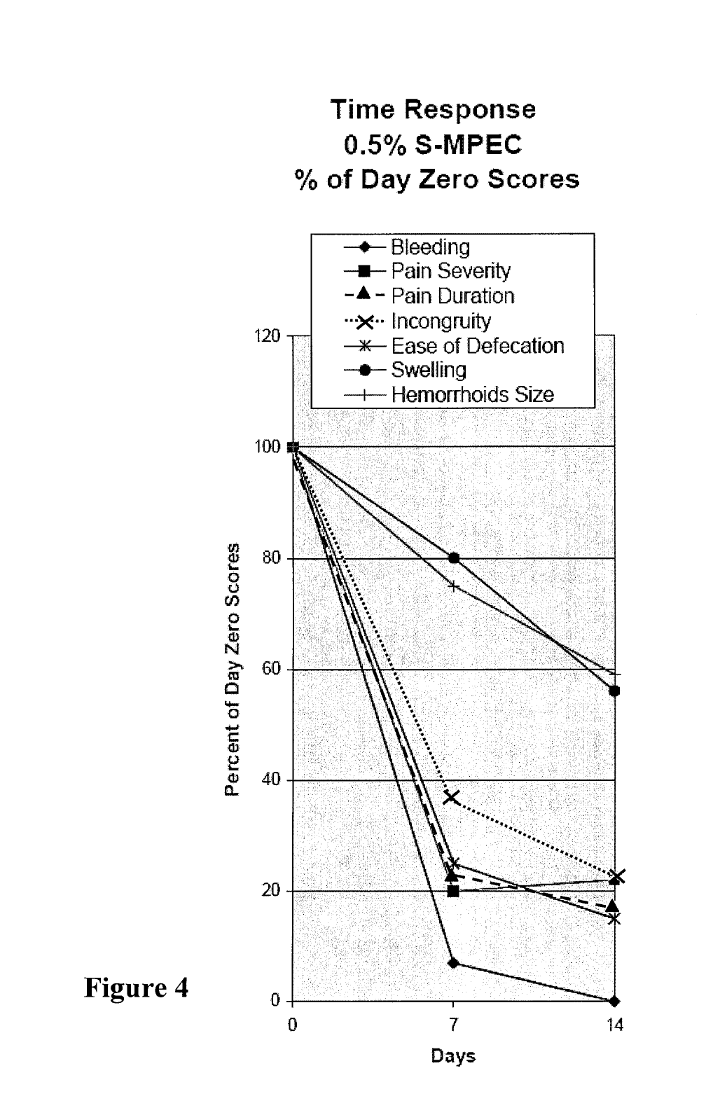

On analysis it was determined that at the start of the study only 50% of patients had subjective symptoms and most patients had grade one or two hemorrhoids (mild) making detecting of differences difficult. Using the visual analog scale (VAS), pain, anal discomfort and pain persistence improved with increasing dose. As for bleeding, a significant difference between the dose levels (p=0.016) and evaluation in the confidence interval of “paired comparison” showed that the 0.5% group was better than the 0.25% or 1.0% groups. By the 14th day of the trial, hemorrhoidal swelling was reduced in the 0.5% (41%) and the 1.0% group (43%). The patient diary review revealed that all symptoms were improved starting on day 1, peaked by day 7 and were maintained to day 14, as shown in FIG. 4. Comparison of doses showed that the 0.5% dose provided the most consistent improvements.

### Example 5

A randomized, double-blinded, placebo controlled dose study of S-MPEC was conducted using 0.25%, 0.5% and 1% concentrations, wherein a 5, 10 or 20 mg dose in the form of two (2) grams was administered in a single dose. The dosage was applied intra-rectally twice a day (b.i.d) for 4 weeks in patients with hemorrhoids.

The primary endpoint of this trial was the reduction in hemorrhoidal area (size) at 4 weeks compared to baseline. Secondary endpoints were subjective symptoms of bleeding, pain, anal discomfort, difficulty in defecation and prolapse rated on a visual analog scale.

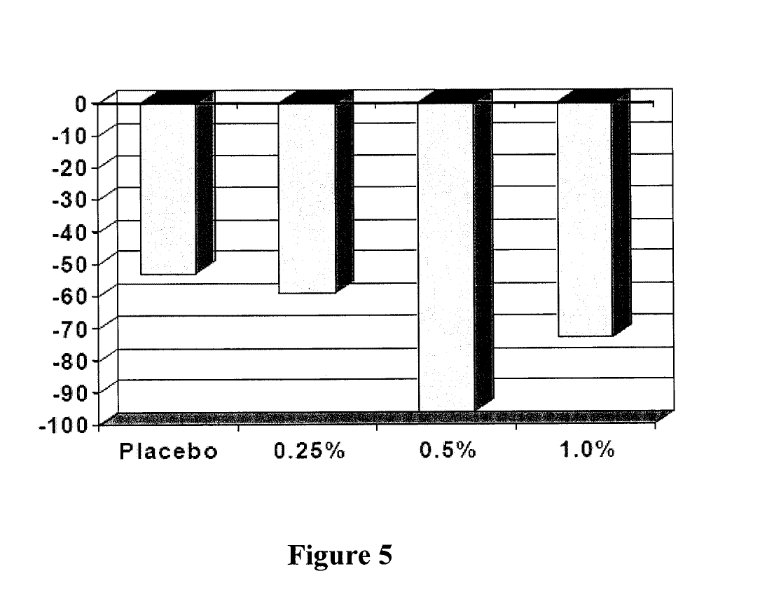

Efficacy Results: The reduction in hemorrhoidal size did not reach statistical significance when comparing the size of a hemorrhoid before administration to the size at the end of study. However, the 0.5% dose was the most effective, as shown in FIG. 5, wherein the hemorrhoid size was greatly reduced relative to the placebo and other concentrations. The difference in hemorrhoid size after 4 weeks compared to placebo had a p-value of 0.076. The slope of the line reflecting the dose-response relationship to 0.5% and 1.0% iferanserin was significantly different from zero. Thus there was a dose-response relationship, with the 0.5% concentration providing the most improvement in hemorrhoid size. Tolerance for the investigational product under the condition of the study was considered good as there was no difference in the incidence of adverse events among the placebo or active dose groups and there was no clinical noteworthy adverse events.

### Example 6

A double-blind placebo-controlled study was conducted to assess the effects of topical iferanserin (S-MPEC) cream (0.5%), applied intra-rectally twice a day for two weeks, on bleeding and other symptoms of patients with internal hemorrhoids (stages 1-3). The primary endpoint of this trial was the change from pre-treatment in patient assessment of hemorrhoid bleeding (10-point scale where 1=no bleeding, 10=extreme bleeding) at the end of one- and two-weeks of treatment. The 0.5% dose was selected based on previous performance, such as shown in Examples 3, 4 and 5 wherein 0.5% concentration consistently performed better then 0.25% and 1% in both patient reported symptoms and in physician rated symptoms at day 7 in a patient population similar to the planned study population. Further, the 0.5% dose was the only dose to approach statistical significance in the primary endpoint (reduction in hemorrhoid size at week 4; p=0.076) (See FIG. 5) with favorable results in bleeding, itching and pain endpoints.

Subjects self-administered placebo or 0.5% iferanserin cream twice-a-day for 14 days and completed a diary evaluating their symptoms (bleeding, ease of bowel movement, pain, prolapse, fullness, throbbing, tenderness and itching) every day during the treatment period. Investigator examinations occurred on days 0 and 14 which included evaluation of hemorrhoidal size, bleeding frequency and intensity, soiling, pruritis, pain, prolapse and incontinence to gas.

One hundred and twenty-one (121) patients were treated, 61 with iferanserin and 60 with placebo. Inclusion criteria required that the main symptom was bleeding episodes of at least every other day during the last two weeks before enrollment in the study. Daily patient dairies for bleeding, itching and pain/discomfort were recorded for 14 day with assessments taken at day 7 and 14 based on a 10 point scale, wherein 1 was used for less symptoms and 10 was for extreme conditions.

Efficacy Results:
(a) Bleeding: Iferanserin caused a reduction in bleeding from Day 1 that continued to the end of treatment. The effect was immediate and lasted for the duration of the study. On Day 1, reduction of bleeding was significantly different from baseline for iferanserin, but not significantly different than placebo. On Day 2, both iferanserin and placebo were significantly different from baseline, but not between each other. From Day 3 onwards, both iferanserin and placebo were statistically significantly different from baseline and iferanserin was significantly different from placebo. In addition statistical significance was observed between iferanserin and placebo groups for the number of subjects demonstrating cessation of bleeding.
(b) Itching: The effect of iferanserin on itching mirrored those of its effects on bleeding, with statistically significant improvement versus placebo from the fourth day of treatment that was maintained throughout the treatment period.
(c) Pain: Iferanserin reduced the level of pain to the almost no symptom level from the third day of treatment, which was significantly different from placebo on many of the remaining days of the study.
(d) Tenderness: The effects of iferanserin in ameliorating tenderness were statistically significant from the second day of treatment and were maintained throughout the trial period.
(e) Ease of Defecation: Iferanserin improved the ease of defecation and was statistically significantly better than baseline from day three to the end of the study.

Safety Results:

There were no serious adverse events reported. There were 42 adverse events reported during the study and were observed to be more prevalent in the placebo group then those on therapy. Based on the findings from these this study, cessation of bleeding appears to be a viable (and more desirable) endpoint than reduction of bleeding. Likewise, all the secondary endpoints are achievable and desirable for a hemorrhoid therapy.

Recurrence of Symptoms

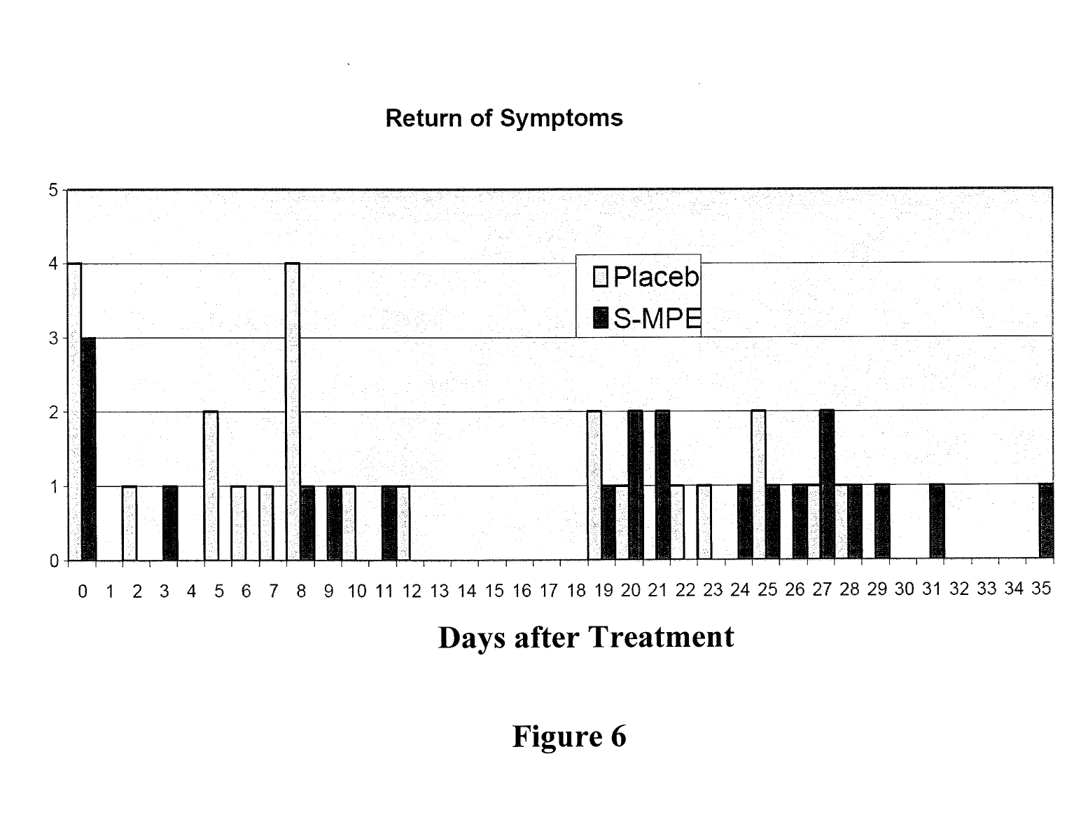

Patients were called 45 days after the end of the treatment period and asked whether they had a recurrence of hemorrhoidal symptoms. In both the iferanserin and the placebo groups 61% of the patients did not experience recurrence of any of the symptoms up to 45 days post-treatment. In those patients who did have a recurrence of symptoms during this time period there was a significant delay (p<0.05) in the appearance of the symptoms after termination of iferanserin treatment as compared to placebo; iferanserin delayed the reappearance of symptoms by an average of 18 days, compared to 12 days with placebo, a 50% difference, as shown in FIG. 6. Thus, not only did iferanserin decrease the major symptoms of hemorrhoids during treatment but it also delayed the time for these symptoms to reappear after termination of treatment. This prolonged symptom-free period, following treatment with iferanserin, provides for less frequent hemorrhoidal episodes, or at least less frequent episodes of hemorrhoidal symptoms in patients who experience chronic hemorrhoids, thereby translating into less frequent treatment.

Hemorrhoidal symptoms can also recur while treatment is continuing. In this study, bleeding, itching and pain were analyzed for recurrence during the 14-day dosing period. In the trial cessation of a symptom was defined as the absence of the symptom for as little as one day; in a posthoc analysis cessation was defined as three consecutive days without the specific symptom. When cessation of bleeding was defined as one day without the symptom, 71% of placebo patients reported that bleeding stopped; 60% of those that stopped for one day had a recurrence of bleeding while still on treatment. For iferanserin, 89% of patients reported cessation of bleeding for at least one day, and of these only 32% reported a recurrence of bleeding.

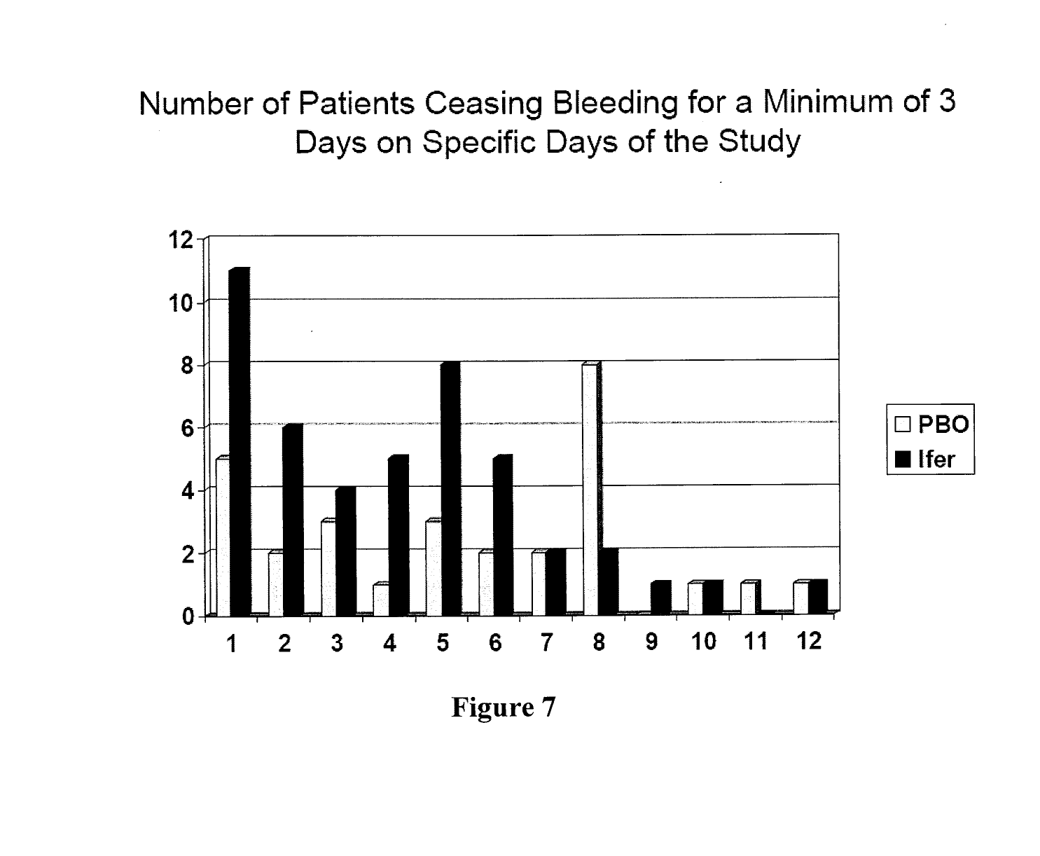

When a stricter definition of bleeding was used, i.e., cessation for three consecutive days, 56 patients of the iferanserin group and 56 patients of the placebo group, who were showing such a symptom, were questioned and 52% of placebo patients (29 patients) reported this effect, while 34% of these reported a recurrence of bleeding. In the iferanserin group, 82% of patients (46 patients) reported stopping of bleeding for three straight days, and of these only 23% indicated that bleeding had resumed. These results, as shown in FIG. 7, indicate that iferanserin can not only delay recurrence of bleeding after treatment has stopped but it also has a major inhibitory effect on recurrence of bleeding during treatment.

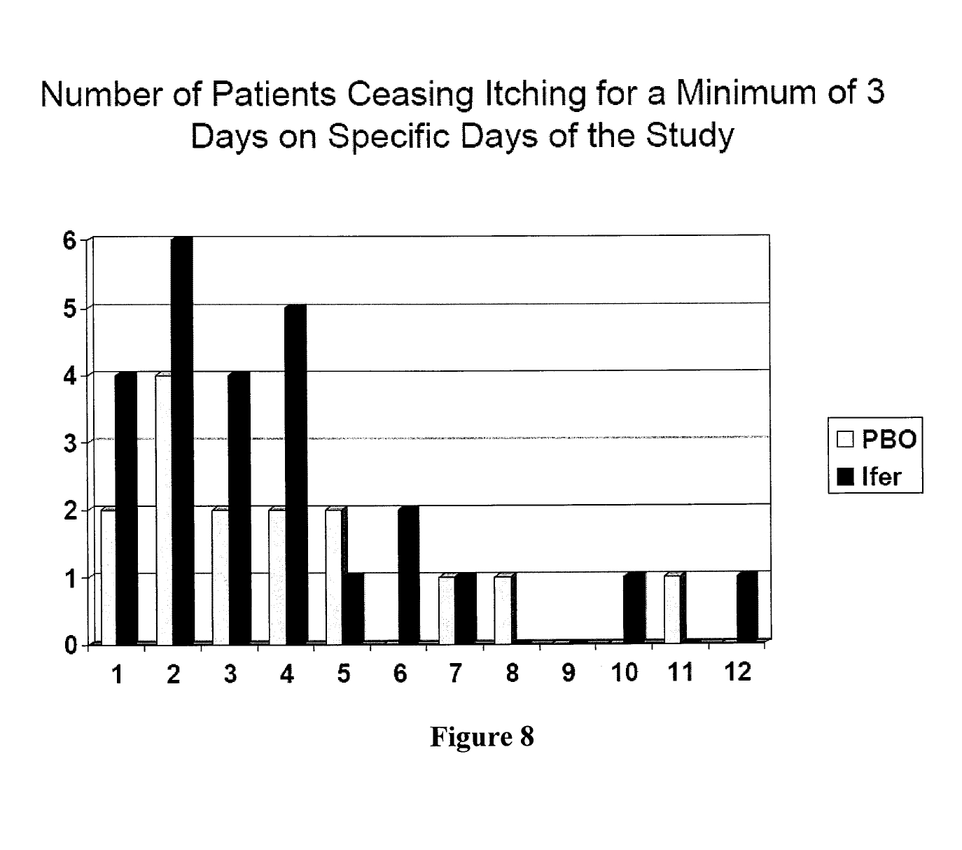

With respect to itching, 66% of placebo-treated patients had cessation when this was defined as one day without the symptom, and of these 37% had a recurrence. This compares to 90% of iferanserin patients who stopped itching for at least one day, and of these 25% had a recurrence. When cessation of itching was defined as three consecutive days of no symptom, 31 patients of the iferanserin group and 29 patients of the placebo group, who were showing such a symptom, were questioned and 52% of placebo patients reported (15 patients) stopping the symptom, while 27% reported a recurrence; for the iferanserin group 80% (25 patients) documented no itching for three consecutive days, and of these there was a recurrence in 16%. The results are shown in FIG. 8.

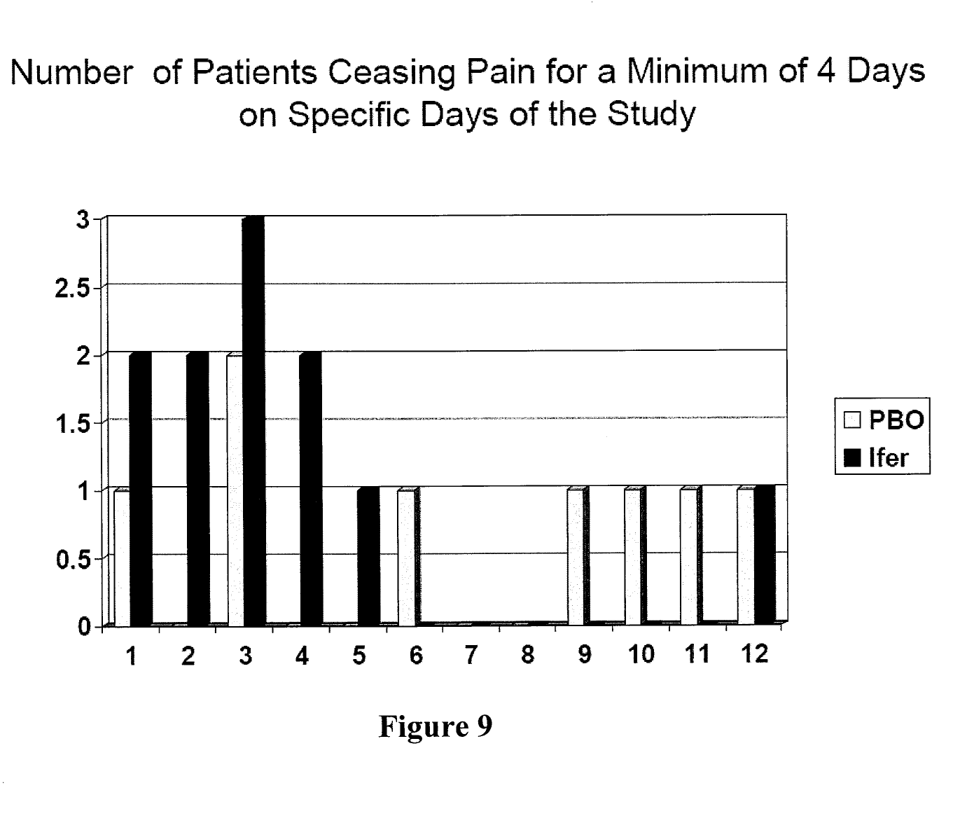

For those patients reporting with pain (a relatively small amount, on the order of 35%), 50% reported at least one pain-free day after treatment with placebo, and 42% of these had a recurrence of pain. In the iferanserin group 72% documented at least one day free of pain, and 33% of these had a recurrence. When cessation of pain was defined as four consecutive days of no pain, 24 patients of the iferanserin group and 18 patients of the placebo group, who were showing such a symptom, were questioned and 33% of placebo patients reported successful treatment, and of these 25% had a recurrence; for the iferanserin group 61% reported three consecutive days of no pain, and of these only 9% had a recurrence of this symptom, as shown in FIG. 9.

The results, as with bleeding, indicate that iferanserin can prevent the recurrence of itching and pain during the treatment period and thereafter. The results of this study shows that topical iferanserin, applied for 14 days, delays the appearance of hemorrhoidal symptoms after termination of treatment and that it also decreases the emergence of symptoms such as bleeding, itching and pain during treatment.

That which is claimed is:

1. A therapeutic composition comprising from 0.3% to 0.7% of S-2′-[2-(1-methyl-2-piperidyl)ethyl]cinnamanilide hydrochloride, wherein the S-2′-[2-(1-methyl-2-piperidyl)ethyl]cinnamanilide hydrochloride is substantially free or devoid of the (R) enantiomer.

2. The therapeutic composition according to claim 1, wherein the composition comprises about 0.5% of S-2′-[2-(1-methyl-2-piperidyl)ethyl]cinnamanilide hydrochloride.

3. The therapeutic composition according to claim 1, wherein the composition consist of 0.5% of S-2′-[2-(1-methyl-2-piperidyl)ethyl]cinnamanilide hydrochloride as the active agent.

4. The therapeutic composition according to claim 1 in the form of a paste, ointment, cream or gel.

5. The therapeutic composition according to claim 2 in the form of a paste, ointment, cream or gel.

6. The therapeutic composition according to claim 1, wherein S-2′-[2-(1-methyl-2-piperidyl)ethyl]cinnamanilide hydrochloride is devoid of any (R) enantiomer.

7. A homogeneous composition for treating internal and/or external hemorrhoids, the composition comprising:
S-2′-[2-(1-methyl-2-piperidyl)ethyl]cinnamanilide hydrochloride in an amount from 0.3% to 0.7%,
liquid paraffin in an amount form about 5% to 20%;
cetanol in an amount from about 2% to about 10%; and
white vaseline to provide for 100%.

8. The homogeneous composition according to claim 7, comprising 0.5% of S-2′-[2-(1-methyl-2-piperidyl)ethyl]cinnamanilide hydrochloride; 10% of liquid paraffin; 6% of cetanol and 83.5% of white vaseline.

9. A method of treating internal and/or external hemorrhoids, the method comprising:
(a) administering a topical composition to the anorectal region of a subject in need of such treatment, wherein the composition comprises from about 0.3% to 0.7% concentration of S-2′-[2-(1-methyl-2-piperidyl)ethyl]cinnamanilide hydrochloride, wherein the S-2′-[2-(1-methyl-2-piperidyl)ethyl]cinnamanilide hydrochloride is substantially free or devoid of the (R) enantiomer; and
(b) repeating step (a) for a period of time ranging from 1 to 14 days.

10. The method according to claim 9, wherein the composition comprises about 0.5% of S-2′-[2-(1-methyl-2-piperidyl)ethyl]cinnamanilide hydrochloride.

11. The method according to claim 9, wherein the composition consist of 0.5% of S-2′-[2-(1-methyl-2-piperidyl)ethyl]cinnamanilide hydrochloride as the active agent.

12. The method according to claim 9, wherein the composition is in the form of a paste, ointment, cream or gel.

13. The method according to claim 9, wherein the composition is administered twice a day for at least 7 days.

14. A method of delaying or inhibiting recurrence of internal and/or external hemorrhoids, the method comprising:
administering a topical composition to the anorectal region of a subject in need of such treatment, wherein the composition comprises from about 0.3% to about 0.7% concentration of S-2′-[2-(1-methyl-2-piperidyl)ethyl]cinnamanilide hydrochloride for at least a period of time ranging from 1 to 14 days, wherein the S-2′-[2-(1-methyl-2-piperidyl)ethyl]cinnamanilide hydrochloride is substantially free or devoid of the (R) enantiomer.

15. The method according to claim 14, wherein the composition comprises about 0.5% of S-2′-[2-(1-methyl-2-piperidyl)ethyl]cinnamanilide hydrochloride.

16. The method according to claim 14, wherein the composition consist of 0.5% of S-2′-[2-(1-methyl-2-piperidyl)ethyl]cinnamanilide hydrochloride as the active agent.

17. The method according to claim 16, wherein the composition is in the form of a paste, ointment, cream or gel.

18. A single dose applicator comprising an ointment, wherein the ointment comprises from about 0.3% to 0.7% concentration of S-2′-[2-(1-methyl-2-piperidyl)ethyl]cinnamanilide, wherein the S-2′-[2-(1-methyl-2-piperidyl)ethyl]cinnamanilide hydrochloride is substantially free or devoid of the (R) enantiomer.

19. The single dose applicator according to claim 18, wherein the composition comprises about 0.5% of S-2′-[2-(1-methyl-2-piperidyl)ethyl]cinnamanilide hydrochloride.

20. The single dose applicator according to claim 18, wherein the composition consist of 0.5% of S-2′-[2-(1-methyl-2-piperidyl)ethyl]cinnamanilide hydrochloride as the active agent.

21. A kit comprising a multiplicity of the single dose applicators according to claim 18.

22. The kit according to claim 21 comprising from a sufficient number of applicators for treatment of hemorrhoids from 7 to 14 days.
